(19) United States
(12) Patent Application Publication
(10) Pub. No.: US 2019/0060883 A1
(43) Pub. Date: Feb. 28, 2019

(54) MAGNETIC PARTICLE-IONIC LIQUID COMPOSITE MATERIALS AND METHODS OF MAKING AND USE THEREOF

(71) Applicants: The Board of Trustees of The University of Alabama, Tuscaloosa, AL (US); Reliance Industries Limited, Navi Mumbai (IN)

(72) Inventors: Robin D. Rogers, Tuscaloosa, AL (US); Rajkumar Kore, Tuscaloosa, AL (US); Anand D. Sawant, Tuscaloosa, AL (US); Pavankumar Aduri, Navi Mumbai (IN)

(21) Appl. No.: 16/113,002

(22) Filed: Aug. 27, 2018

Related U.S. Application Data
(60) Provisional application No. 62/551,856, filed on Aug. 30, 2017.

(51) Int. Cl. B01J 27/125; C07C 2/86; B01J 23/745; B01J 35/02; B01J 35/00

(52) U.S. Cl. CPC B01J 27/125; C07C 2/861; B01J 23/745; C07C 2601/16; B01J 35/0033; C07C 2523/745; C07C 2527/126; B01J 35/026

(57) ABSTRACT

Described herein are magnetic particle-ionic liquid composite materials, and methods of making and use thereof. The magnetic particle-ionic liquid composite materials can comprise an ionic liquid conjugated to a magnetic particle, wherein the ionic liquid comprises at least one cation and at least one metal halide anion; and wherein the ionic liquid is not covalently bound to the magnetic particle.

### CROSS-REFERENCE TO RELATED APPLICATIONS

This application claims the benefit of U.S. Provisional Application No. 62/551,856, filed Aug. 30, 2017, which is hereby incorporated herein by reference in its entirety.

### BACKGROUND

Conventional catalysts for Lewis acid catalyzed reactions, such as alkylation reactions, include HF/AlCl₃, which is toxic, corrosive, and cannot be reused. Other conventional catalysts include solid acid catalysts, such as zeolites, but these catalysts lack efficiency. An additional catalyst includes ionic liquids supported on silica, but the active hydroxyl groups on silica surfaces deactivate the ionic liquid catalyst during impregnation. Covalent bonding (e.g., grafting) of the ionic liquid catalyst on the silica support has also been used, but the grafting increases the cost of the catalyst. Accordingly, an inexpensive, efficient, non-toxic, recyclable catalyst for Lewis acid catalyzed reactions is still needed. The magnetic particle-ionic liquid composite materials discussed herein address these and other needs.

### SUMMARY

In accordance with the purposes of the disclosed systems and methods, as embodied and broadly described herein, the disclosed subject matter relates to magnetic particle-ionic liquid composite materials.

Additional advantages of the disclosed systems and methods will be set forth in part in the description which follows, and in part will be obvious from the description. The advantages of the disclosed systems and methods will be realized and attained by means of the elements and combinations particularly pointed out in the appended claims. It is to be understood that both the foregoing general description and the following detailed description are exemplary and explanatory only and are not restrictive of the disclosed systems and methods, as claimed.

The details of one or more embodiments of the invention are set forth in the accompanying drawings and the description below. Other features, objects, and advantages of the invention will be apparent from the description and drawings, and from the claims.

### BRIEF DESCRIPTION OF THE FIGURES

The accompanying figures, which are hereby incorporated in and constitute a part of this specification, illustrate several aspects of the disclosure, and together with the description, serve to explain the principles of the disclosure.

FIG. 1 shows the preparation of [HN₂₂₂][Al₂Cl₇] ionic liquid loaded Fe₃O₄ nanoparticles.

FIG. 2 shows a portion of the infrared (IR) spectra of 20 wt % [HN₂₂₂][Al₂Cl₇] loaded Fe₃O₄ nanoparticles compared with neat Fe₃O₄ nanoparticles, [HN₂₂₂][Al₂Cl₇] and [HN₂₂₂][AlCl₄] and recycled [HN₂₂₂][Al₂Cl₇] loaded Fe₃O₄ nanoparticles.

FIG. 3 shows a portion of the infrared (IR) spectra of 20 wt % [HN₂₂₂][Al₂Cl₇] loaded Fe₃O₄ nanoparticles compared with neat Fe₃O₄ nanoparticles, [HN₂₂₂][Al₂Cl₇] and [HN₂₂₂][AlCl₄] and recycled [HN₂₂₂][Al₂Cl₇] loaded Fe₃O₄ nanoparticles.

FIG. 4 shows a portion of the infrared (IR) spectra of 20 wt % [HN₂₂₂][Al₂Cl₇] loaded Fe₃O₄ nanoparticles compared with neat Fe₃O₄ nanoparticles, [HN₂₂₂][Al₂Cl₇] and [HN₂₂₂][AlCl₄] and recycled [HN₂₂₂][Al₂Cl₇] loaded Fe₃O₄ nanoparticles.

FIG. 5 shows a scanning electron microscopy (SEM) image of Fe₃O₄ nanoparticles.

FIG. 6 shows a SEM image of fresh 20 wt % [HN₂₂₂][Al₂Cl₇] loaded Fe₃O₄ nanoparticles.

FIG. 7 shows a SEM image of 20 wt % [HN₂₂₂][Al₂Cl₇] loaded Fe₃O₄ nanoparticles after being recycled for six runs.

FIG. 8 shows the energy dispersive X-ray spectrum (EDX) of fresh 20 wt % [HN₂₂₂][Al₂Cl₇] loaded Fe₃O₄ nanoparticles.

FIG. 9 shows the energy dispersive X-ray spectrum (EDX) of 20 wt % [HN₂₂₂][Al₂Cl₇] loaded Fe₃O₄ nanoparticles after being recycled for six runs.

FIG. 10 shows the N₂-adsorption isotherm of 20 wt % of IL loaded Fe₃O₄ nanoparticles compared with Fe₃O₄ nanoparticles.

### DETAILED DESCRIPTION

The materials, compositions, articles, and methods described herein can be understood more readily by reference to the following detailed description of specific aspects of the disclosed subject matter and the Examples and Figures included therein.

Before the present materials, compositions, articles, devices, and methods are disclosed and described, it is to be understood that the aspects described below are not limited to specific synthetic methods or specific reagents, as such may, of course, vary. It is also to be understood that the terminology used herein is for the purpose of describing particular aspects only and is not intended to be limiting.

Also, throughout this specification, various publications are referenced. The disclosures of these publications in their entireties are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

### General Definitions

In this specification and in the claims that follow, reference will be made to a number of terms, which shall be defined to have the following meanings:

Throughout the description and claims of this specification the word “comprise” and other forms of the word, such as “comprising” and “comprises,” means including but not limited to, and is not intended to exclude, for example, other additives, components, integers, or steps.

As used in the description and the appended claims, the singular forms “a,” “an,” and “the” include plural referents unless the context clearly dictates otherwise. Thus, for example, reference to “a composition” includes mixtures of two or more such compositions, reference to “an ionic liquid” includes mixtures of two or more such ionic liquids, reference to “the compound” includes mixtures of two or more such compounds, and the like.

“Optional” or “optionally” means that the subsequently described event or circumstance can or cannot occur, and that the description includes instances where the event or circumstance occurs and instances where it does not.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed, then “less than or equal to” the value, “greater than or equal to the value,” and possible ranges between values are also disclosed, as appropriately understood by the skilled artisan. For example, if the value “10” is disclosed, then “less than or equal to 10” as well as “greater than or equal to 10” is also disclosed. It is also understood that throughout the application data are provided in a number of different formats and that this data represent endpoints and starting points and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Chemical Definitions

Terms used herein will have their customary meaning in the art unless specified otherwise. The organic moieties mentioned when defining variable positions within the general formulae described herein (e.g., the term “halogen”) are collective terms for the individual substituents encompassed by the organic moiety. The prefix Cₙ-Cₘ preceding a group or moiety indicates, in each case, the possible number of carbon atoms in the group or moiety that follows.

References in the specification and concluding claims to the molar ratio of a particular element or component in a composition denotes the molar relationship between the element or component and any other elements or components in the composition or article for which a part by weight is expressed. Thus, in a compound containing 2 moles of X and 5 moles of Y, X and Y are present at a molar ratio of 2:5, and are present in such ratio regardless of whether additional components are contained in the compound.

A weight percent (wt. %) of a component, unless specifically stated to the contrary, is based on the total weight of the formulation or composition in which the component is included.

The term “ion,” as used herein, refers to any molecule, portion of a molecule, cluster of molecules, molecular complex, moiety, or atom that contains a charge (positive, negative, or both at the same time within one molecule, cluster of molecules, molecular complex, or moiety (e.g., zwitterions)) or that can be made to contain a charge. Methods for producing a charge in a molecule, portion of a molecule, cluster of molecules, molecular complex, moiety, or atom are disclosed herein and can be accomplished by methods known in the art, e.g., protonation, deprotonation, oxidation, reduction, alkylation, acetylation, esterification, deesterification, hydrolysis, etc.

The term “anion” is a type of ion and is included within the meaning of the term “ion.” An “anion” is any molecule, portion of a molecule (e.g., zwitterion), cluster of molecules, molecular complex, moiety, or atom that contains a net negative charge or that can be made to contain a net negative charge. The term “anion precursor” is used herein to specifically refer to a molecule that can be converted to an anion via a chemical reaction (e.g., deprotonation).

The term “cation” is a type of ion and is included within the meaning of the term “ion.” A “cation” is any molecule, portion of a molecule (e.g., zwitterion), cluster of molecules, molecular complex, moiety, or atom, that contains a net positive charge or that can be made to contain a net positive charge. The term “cation precursor” is used herein to specifically refer to a molecule that can be converted to a cation via a chemical reaction (e.g., protonation or alkylation).

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, heteroatoms present in a compound or moiety, such as nitrogen, can have hydrogen substituents and/or any permissible substituents of organic compounds described herein which satisfy the valency of the heteroatom. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound (e.g., a compound that does not spontaneously undergo transformation such as by rearrangement, cyclization, elimination, etc.

“Z¹,” “Z²,” “Z³,” and “Z⁴” are used herein as generic symbols to represent various specific substituents. These symbols can be any substituent, not limited to those disclosed herein, and when they are defined to be certain substituents in one instance, they can, in another instance, be defined as some other substituents.

As used herein, the term “alkyl” refers to saturated, straight-chained or branched saturated hydrocarbon moieties. Unless otherwise specified, C₁-C₅₀ (e.g., C₁-C₄₅, C₁-C₄₀, C₁-C₃₅, C₁-C₃₀, C₁-C₂₅, C₁-C₂₀, C₁-C₁₈, C₁-C₁₆, C₁-C₁₄, C₁-C₁₂, C₁-C₁₀, C₁-C₈, C₁-C₆, or C₁-C₄) alkyl groups are intended. Examples of alkyl groups include methyl, ethyl, propyl, 1-methyl-ethyl, butyl, 1-methyl-propyl, 2-methyl-propyl, 1,1-dimethyl-ethyl, pentyl, 1-methyl-butyl, 2-methyl-butyl, 3-methyl-butyl, 2,2-dimethyl-propyl, 1-ethyl-propyl, hexyl, 1,1-dimethyl-propyl, 1,2-dimethyl-propyl, 1-methyl-pentyl, 2-methyl-pentyl, 3-methyl-pentyl, 4-methyl-pentyl, 1,1-dimethyl-butyl, 1,2-dimethyl-butyl, 1,3-dimethyl-butyl, 2,2-dimethyl-butyl, 2,3-dimethyl-butyl, 3,3-dimethyl-butyl, 1-ethyl-butyl, 2-ethyl-butyl, 1,1,2-trimethyl-propyl, 1,2,2-trimethyl-propyl, 1-ethyl-1-methyl-propyl, and 1-ethyl-2-methyl-propyl. Alkyl substituents may be unsubstituted or substituted with one or more chemical moieties. The alkyl group can be substituted with one or more groups including, but not limited to, hydroxy, halogen, acyl, alkyl, alkoxy, alkenyl, alkynyl, aryl, heteroaryl, aldehyde, amino, cyano, carboxylic acid, ester, ether, ketone, nitro, phosphonyl, silyl, sulfo-oxo, sulfonyl, sulfone, sulfoxide, or thiol, as described below, provided that the substituents are sterically compatible and the rules of chemical bonding and strain energy are satisfied.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” specifically refers to an alkyl group that is substituted with one or more halides (halogens; e.g., fluorine, chlorine, bromine, or iodine). The term “alkoxyalkyl” specifically refers to an alkyl group that is substituted with one or more alkoxy groups, as described below. The term “alkylamino” specifically refers to an alkyl group that is substituted with one or more amino groups, as described below, and the like. When “alkyl” is used in one instance and a specific term such as “alkylalcohol” is used in another, it is not meant to imply that the term “alkyl” does not also refer to specific terms such as “alkylalcohol” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

As used herein, the term “alkenyl” refers to unsaturated, straight-chained, or branched hydrocarbon moieties containing a double bond. Unless otherwise specified, C₂-C₅₀ (e.g., C₂-C₄₅, C₂-C₄₀, C₂-C₃₅, C₂-C₃₀, C₂-C₂₅, C₂-C₂₀, C₂-C₁₈, C₂-C₁₆, C₂-C₁₄, C₂-C₁₂, C₂-C₁₀, C₂-C₈, C₂-C₆, or C₂-C₄) alkenyl groups are intended. Alkenyl groups may contain more than one unsaturated bond. Examples include ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-diethyl-1-butenyl, 3,3-diethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl, and 1-ethyl-2-methyl-2-propenyl. The term “vinyl” refers to a group having the structure —CH═CH₂; 1-propenyl refers to a group with the structure —CH═CH—CH₃; and 2-propenyl refers to a group with the structure —CH₂—CH═CH₂. Asymmetric structures such as (Z¹Z²)C═C(Z³Z⁴) are intended to include both the E and Z isomers. This can be presumed in structural formulae herein wherein an asymmetric alkene is present, or it can be explicitly indicated by the bond symbol C═C. Alkenyl substituents may be unsubstituted or substituted with one or more chemical moieties. Examples of suitable substituents include, for example, alkyl, halogenated alkyl, alkoxy, alkenyl, alkynyl, aryl, heteroaryl, acyl, aldehyde, amino, cyano, carboxylic acid, ester, ether, halide, hydroxy, ketone, nitro, phosphonyl, silyl, sulfo-oxo, sulfonyl, sulfone, sulfoxide, or thiol, as described below, provided that the substituents are sterically compatible and the rules of chemical bonding and strain energy are satisfied.

As used herein, the term “alkynyl” represents straight-chained or branched hydrocarbon moieties containing a triple bond. Unless otherwise specified, C₂-C₅₀ (e.g., C₂-C₄₅, C₂-C₄₀, C₂-C₃₅, C₂-C₃₀, C₂-C₂₅, C₂-C₂₀, C₂-C₁₈, C₂-C₁₆, C₂-C₁₄, C₂-C₁₂, C₂-C₁₀, C₂-C₈, C₂-C₆, or C₂-C₄) alkynyl groups are intended. Alkynyl groups may contain more than one unsaturated bond. Examples include C₂-C₆-alkynyl, such as ethynyl, 1-propynyl, 2-propynyl (or propargyl), 1-butynyl, 2-butynyl, 3-butynyl, 1-methyl-2-propynyl, 1-pentynyl, 2-pentynyl, 3-pentynyl, 4-pentynyl, 3-methyl-1-butynyl, 1-methyl-2-butynyl, 1-methyl-3-butynyl, 2-methyl-3-butynyl, 1,1-dimethyl-2-propynyl, 1-ethyl-2-propynyl, 1-hexynyl, 2-hexynyl, 3-hexynyl, 4-hexynyl, 5-hexynyl, 3-methyl-1-pentynyl, 4-methyl-1-pentynyl, 1-methyl-2-pentynyl, 4-methyl-2-pentynyl, 1-methyl-3-pentynyl, 2-methyl-3-pentynyl, 1-methyl-4-pentynyl, 2-methyl-4-pentynyl, 3-methyl-4-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl, and 1-ethyl-1-methyl-2-propynyl. Alkynyl substituents may be unsubstituted or substituted with one or more chemical moieties. Examples of suitable substituents include, for example, alkyl, halogenated alkyl, alkoxy, alkenyl, alkynyl, aryl, heteroaryl, acyl, aldehyde, amino, cyano, carboxylic acid, ester, ether, halide, hydroxy, ketone, nitro, phosphonyl, silyl, sulfo-oxo, sulfonyl, sulfone, sulfoxide, or thiol, as described below.

As used herein, the term “aryl,” as well as derivative terms such as aryloxy, refers to groups that include a monovalent aromatic carbocyclic group of from 3 to 50 carbon atoms. Aryl groups can include a single ring or multiple condensed rings. In some embodiments, aryl groups include C₆-C₁₀ aryl groups. Examples of aryl groups include, but are not limited to, phenyl, biphenyl, naphthyl, tetrahydronaphthyl, phenylcyclopropyl, and indanyl. In some embodiments, the aryl group can be a phenyl, indanyl or naphthyl group. The term “heteroaryl” is defined as a group that contains an aromatic group that has at least one heteroatom incorporated within the ring of the aromatic group. Examples of heteroatoms include, but are not limited to, nitrogen, oxygen, sulfur, and phosphorus. The term “non-heteroaryl,” which is included in the term “aryl,” defines a group that contains an aromatic group that does not contain a heteroatom. The aryl or heteroaryl substituents may be unsubstituted or substituted with one or more chemical moieties. Examples of suitable substituents include, for example, alkyl, halogenated alkyl, alkoxy, alkenyl, alkynyl, aryl, heteroaryl, acyl, aldehyde, amino, cyano, carboxylic acid, ester, ether, halide, hydroxy, ketone, nitro, phosphonyl, silyl, sulfo-oxo, sulfonyl, sulfone, sulfoxide, or thiol as described herein. The term “biaryl” is a specific type of aryl group and is included in the definition of aryl. Biaryl refers to two aryl groups that are bound together via a fused ring structure, as in naphthalene, or are attached via one or more carbon-carbon bonds, as in biphenyl.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, etc. The term “heterocycloalkyl” is a cycloalkyl group as defined above where at least one of the carbon atoms of the ring is substituted with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, alkoxy, alkenyl, alkynyl, aryl, heteroaryl, acyl, aldehyde, amino, cyano, carboxylic acid, ester, ether, halide, hydroxy, ketone, nitro, phosphonyl, silyl, sulfo-oxo, sulfonyl, sulfone, sulfoxide, or thiol as described herein.

The term “cycloalkenyl” as used herein is a non-aromatic carbon-based ring composed of at least three carbon atoms and containing at least one double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, and the like. The term “heterocycloalkenyl” is a type of cycloalkenyl group as defined above, and is included within the meaning of the term “cycloalkenyl,” where at least one of the carbon atoms of the ring is substituted with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, alkoxy, alkenyl, alkynyl, aryl, heteroaryl, acyl, aldehyde, amino, cyano, carboxylic acid, ester, ether, halide, hydroxy, ketone, nitro, phosphonyl, silyl, sulfo-oxo, sulfonyl, sulfone, sulfoxide, or thiol as described herein.

The term “cyclic group” is used herein to refer to either aryl groups, non-aryl groups (i.e., cycloalkyl, heterocycloalkyl, cycloalkenyl, and heterocycloalkenyl groups), or both. Cyclic groups have one or more ring systems that can be substituted or unsubstituted. A cyclic group can contain one or more aryl groups, one or more non-aryl groups, or one or more aryl groups and one or more non-aryl groups.

The term “acyl” as used herein is represented by the formula —C(O)Z¹ where Z¹ can be a hydrogen, hydroxyl, alkoxy, alkyl, halogenated alkyl, alkenyl, alkynyl, aryl, heteroaryl, cycloalkyl, cycloalkenyl, heterocycloalkyl, or heterocycloalkenyl group described above. As used herein, the term “acyl” can be used interchangeably with “carbonyl.” Throughout this specification “C(O)” or “CO” is a short hand notation for C═O.

As used herein, the term “alkoxy” refers to a group of the formula Z¹—O—, where Z¹ is unsubstituted or substituted alkyl as defined above. Unless otherwise specified, alkoxy groups wherein Z¹ is a C₁-C₅₀ (e.g., C₁-C₄₅, C₁-C₄₀, C₁-C₃₅, C₁-C₃₀, C₁-C₂₅, C₁-C₂₀, C₁-C₁₈, C₁-C₁₆, C₁-C₁₄, C₁-C₁₂, C₁-C₁₀, C₁-C₈, C₁-C₆, C₁-C₄) alkyl group are intended. Examples include methoxy, ethoxy, propoxy, 1-methyl-ethoxy, butoxy, 1-methyl-propoxy, 2-methyl-propoxy, 1,1-dimethyl-ethoxy, pentoxy, 1-methyl-butyloxy, 2-methyl-butoxy, 3-methyl-butoxy, 2,2-di-methyl-propoxy, 1-ethyl-propoxy hexoxy, 1,1-dimethyl-propoxy, 1,2-dimethyl-propoxy, 1-methyl-pentoxy, 2-methyl-pentoxy, 3-methyl-pentoxy, 4-methyl-pentoxy, 1,1-dimethyl-butoxy, 1,2-dimethyl-butoxy, 1,3-dimethyl-butoxy, 2,2-dimethyl-butoxy, 2,3-dimethyl-butoxy, 3,3-dimethyl-butoxy, 1-ethyl-butoxy, 2-ethylbutoxy, 1,1,2-trimethyl-propoxy, 1,2,2-trimethyl-propoxy, 1-ethyl-1-methyl-propoxy, and 1-ethyl-2-methyl-propoxy.

The term “aldehyde” as used herein is represented by the formula —C(O)—H.

The terms “amine” or “amino” as used herein are represented by the formula —NZ¹Z², where Z¹ and Z² can each be substitution group as described herein, such as hydrogen, an alkyl, halogenated alkyl, alkenyl, alkynyl, aryl, heteroaryl, cycloalkyl, cycloalkenyl, heterocycloalkyl, or heterocycloalkenyl group described above. “Amido” is —C(O)NZ¹Z².

The term “carboxylic acid” as used herein is represented by the formula —C(O)OH. A “carboxylate” or “carboxyl” group as used herein is represented by the formula —C(O)O⁻.

The term “cyano” as used herein is represented by the formula —CN.

The term “ester” as used herein is represented by the formula —OC(O)Z¹ or —C(O)OZ¹, where Z¹ can be an alkyl, halogenated alkyl, alkenyl, alkynyl, aryl, heteroaryl, cycloalkyl, cycloalkenyl, heterocycloalkyl, or heterocycloalkenyl group described above.

The term “ether” as used herein is represented by the formula Z¹OZ², where Z¹ and Z² can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “ketone” as used herein is represented by the formula Z¹C(O)Z², where Z¹ and Z² can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “halide” or “halogen” or “halo” as used herein refers to fluorine, chlorine, bromine, and iodine.

The term “hydroxyl” as used herein is represented by the formula —OH.

The term “nitro” as used herein is represented by the formula —NO₂.

The term “phosphonyl” is used herein to refer to the phospho-oxo group represented by the formula —P(O)(OZ¹)₂, where Z³ can be hydrogen, an alkyl, halogenated alkyl, alkenyl, alkynyl, aryl, heteroaryl, cycloalkyl, cycloalkenyl, heterocycloalkyl, or heterocycloalkenyl group described above.

The term “silyl” as used herein is represented by the formula —SiZ¹Z²Z³, where Z¹, Z², and Z³ can be, independently, hydrogen, alkyl, halogenated alkyl, alkoxy, alkenyl, alkynyl, aryl, heteroaryl, cycloalkyl, cycloalkenyl, heterocycloalkyl, or heterocycloalkenyl group described above.

The term “sulfonyl” is used herein to refer to the sulfo-oxo group represented by the formula —S(O)₂Z¹, where Z¹ can be hydrogen, an alkyl, halogenated alkyl, alkenyl, alkynyl, aryl, heteroaryl, cycloalkyl, cycloalkenyl, heterocycloalkyl, or heterocycloalkenyl group described above.

The term “sulfonylamino” or “sulfonamide” as used herein is represented by the formula —S(O)₂NH—.

The term “thiol” as used herein is represented by the formula —SH.

The term “sulfide” as used herein is comprises the formula —S—.

As used herein, Me refers to a methyl group; OMe refers to a methoxy group and i-Pr refers to an isopropyl group.

“R¹,” “R²,” “R³,” “Rⁿ,” etc., where n is some integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an amine group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

The term “conjugate” is used to describe a proximate association between two or more chemical components. The components can be covalently bound to one another directly or through a linker moiety, as in a covalent conjugate, or the components can be non-covalently bound to one another, as in a non-covalent conjugate. Non-covalent conjugates have chemical components proximately associated through electrostatic forces (e.g., ionic bonds, dative bonds, van der Waals forces, and the like.)

Unless stated to the contrary, a formula with chemical bonds shown only as solid lines and not as wedges or dashed lines contemplates each possible stereoisomer or mixture of stereoisomer (e.g., each enantiomer, each diastereomer, each meso compound, a racemic mixture, or scalemic mixture).

Reference will now be made in detail to specific aspects of the disclosed materials, compounds, compositions, articles, and methods, examples of which are illustrated in the accompanying Examples and Figures.

Composite Materials

Described herein are magnetic particle-ionic liquid composite materials comprising an ionic liquid conjugated to a magnetic particle.

The ionic liquid can be non-covalently conjugated to the magnetic particle. In some examples, the ionic liquid is conjugated to the magnetic particle by electrostatic forces between the ionic liquid and the magnetic particle. In some examples, in the disclosed magnetic particle-ionic liquid composite material, the magnetic particle is not coated with silica or a silane. In further examples, the at least one cation of the ionic liquid is not covalently bound to the magnetic particle.

In some examples, the magnetic particle-ionic liquid composite material comprises 1 wt % or more of the ionic liquid, based on the total weight of the magnetic particle-ionic liquid composite (e.g., 2 wt % or more, 3 wt % or more, 4 wt % or more, 5 wt % or more, 10 wt % or more, 15 wt % or more, 20 wt % or more, 25 wt % or more, or 30 wt % or more). In some examples, the magnetic particle-ionic liquid composite material comprises 35 wt % or less of the ionic liquid, based on the total weight of the magnetic particle-ionic liquid composite (e.g., 30 wt % or less, 25 wt % or less, 20 wt % or less, 15 wt % or less, 10 wt % or less, 5 wt % or less, 4 wt % or less, 3 wt % or less, or 2 wt % or less). The amount of ionic liquid in the magnetic particle-ionic liquid composite can range from any of the minimum values described above to any of the maximum values described above. For example, the magnetic particle-ionic liquid composite material can comprise from 1 wt % to 35 wt % of the ionic liquid, based on the total weight of the magnetic particle-ionic liquid composite material (e.g., from 1 wt % to 30 wt %, from 1 wt % to 25 wt %, from 1 wt % to 20 wt %, from 1 wt % to 15 wt %, from 1 wt % to 10 wt %, or from 10 wt % to 35 wt %).

Ionic Liquid

In some examples, the ionic liquid comprises at least one cation and at least one metal halide anion, as described herein.

The term “ionic liquid” has many definitions in the art, but is used herein to refer to salts (i.e., compositions comprising cations and anions) that are liquid at a temperature of at or below about 150° C., e.g., at or below about 120, 100, 80, 60, 40, or 25° C. That is, at one or more temperature ranges or points at or below about 150° C. the disclosed ionic liquid compositions are liquid; although, it is understood that they can be solids at other temperature ranges or points. An ionic liquid is not considered a mere solution containing ions as solutes dissolved therein.

The use of the term “liquid” to describe the disclosed ionic liquid component is meant to describe a generally amorphous, non-crystalline, or semi-crystalline state. For example, while some structured association and packing of cations and anions can occur at the atomic level, the disclosed ionic liquids have minor amounts of such ordered structures and are therefore not crystalline solids. The ionic liquids disclosed herein can be fluid and free-flowing liquids or amorphous solids such as glasses or waxes at a temperature at or below about 150° C. In particular examples disclosed herein, the disclosed ionic liquids are liquid at which the composition is applied (i.e., ambient temperature).

Further, the disclosed ionic liquid components are materials composed of at least two different ions; each of which can independently and simultaneously introduce a specific characteristic to the composition not easily obtainable with traditional dissolution and formulation techniques. Thus, by providing different ions and ion combinations, one can change the characteristics or properties of the disclosed ionic liquids in a way not seen by simply preparing various crystalline salt forms. Examples of characteristics that can be controlled in the disclosed ionic liquids include, but are not limited to, melting, solubility control, and rate of dissolution. It is this multi-nature/functionality of the disclosed ionic liquids which allows one to fine-tune or design in very specific desired material properties.

As disclosed herein, the ionic liquids can include at least one metal halide anion. In some embodiments, the disclosed ionic liquids can include two or more anions (e.g., 3, 4, 5, 6, 7, 8, 9, 10, or more anions). The anions in the disclosed ionic liquids can be the same or different. In some aspects, the anions in the disclosed ionic liquids can be different, that is, the ionic liquids can comprise more than one kind of anion (e.g., 2, 3, 4, 5, 6, 7, 8, 9, 10, or more different kinds of anions).

The anions in the disclosed ionic liquids can each be independently selected from metal halide anions (also referred to herein as “halometallates”). The term “metal halide anion” as used herein refers to a complex poly atomic anion, which contain at least a halogen bonded to a primary metal. These complexes may have a number of halogen atoms bonded to the primary metal in excess of the usual valence number of the metal. Alternatively, one or more of such halogen can be replaced by oxygen or other atoms. The term “primary metal” is used to refer to a metal that can form a complex anion with a halogen. In some embodiments, the ionic liquid can include at least two metal halide anions.

In some embodiments, the primary metal in the metal halide anions can include a metal selected from Group II or Group III of the periodic table, transition metals, or combinations thereof. In some examples, the primary metal can be selected from aluminum, iron, chromium, zinc, copper, tin, titanium, palladium, zirconium, gallium, and combinations thereof. In some examples, the at least one metal halide anion comprises an aluminum halide.

The metal halide anions disclosed herein can include at least one halide selected from Cl, F, Br, and I. In some examples, the at least one metal halide anion comprises a metal chloride.

In some examples, the at least one metal halide anion in the ionic liquids disclosed herein can be selected from chloroaluminate, chlorozincate, chloroferrate, chlorogallate, chlorostannate, chloroindate, chlorochromate, chlorocuprate, chlorotitannate, chlorozirconate, chloropalladate, and combinations thereof. In some examples, the at least one metal halide anion can comprise chloroaluminate. In some examples, the at least one metal halide anion can comprise [Al₂Cl₇]⁻.

In some embodiments, the ionic liquid can include at least two metal halide anions. In some examples, the at least two metal halide anions in the ionic liquids disclosed herein can be independently selected from chloroaluminate, chlorozincate, chloroferrate, chlorogallate, chlorostannate, chloroindate, chlorochromate, chlorocuprate, chlorotitannate, chlorozirconate, chloropalladate, and combinations thereof.

The at least two metal halide anions can be incorporated into the ionic liquids in any suitable molar ratio so long as there is a balance of charge with the cation(s). For example, if a singly charged cation is selected (C₁), and two singly charged metal halide anions are selected (M₁Xₐ and M₂Y_{b}), they can be used in an amount that would give an ionic liquid with the following formula: [C₁][M₁Xₐ]_{0.5}[M₂Y_{b}]_{0.5}, where C₁ is a cation; M₁ and M₂ are metals; and Xₐ and Y_{b} are halides. This can indicate that half of the anions can be comprised of the first metal halide and half of the anions can be comprised of the second metal halide. Other examples would include [C₁][M₁Xₐ]_{0.25}[M₂Y_{b}]_{0.75} [C₁][M₁Xₐ]_{0.1}[M₂Y_{b}]_{0.9} and the like. Also, a greater number of different anions can be paired with a properly selected cation, such as a 1 to 1 to 2 ratio of the first metal halide to the second metal halide to a third anion (such as, [C₁][M₁Xₐ]_{0.25}[M₂Y_{b}]_{0.25}[M₃Z_{c}]_{0.5}).

As disclosed herein, the ionic liquids can include at least one cation. For example, the disclosed ionic liquids can comprise one or more cations (e.g., 2, 3, 4, 5, 6, 7, 8, 9, 10, or more different cations). The cations in the disclosed ionic liquids can be the same or different. In some aspects, the cations in the disclosed ionic liquids can be different, that is, the ionic liquids can comprise more than one kind of cation (e.g., 2, 2, 3, 4, 5, 6, 7, 8, 9, 10, or more different kinds of cations).

The cation in the disclosed ionic liquids can be an organic group-containing cation (also referred to herein as “organic cation”). The organic cation can be a complex polyatomic cation, which contains at least an organic group bonded to a heteroatom. In some embodiments, the ionic liquid can include at least one organic cation.

Particular examples of organic cations that can be present in the disclosed ionic liquids include compounds that contain one or more heteroatoms (e.g., nitrogen, phosphorus, oxygen, or sulfur heteroatom(s)). For example, the organic cation can comprise a linear, branched, or cyclic compound comprising one or more heteroatoms.

Nitrogen atom-containing groups can exist as a neutral compound or can be converted to a positively-charged quaternary ammonium species, for example, through alkylation or protonation of the nitrogen atom. Thus, compounds that possess a quaternary nitrogen atom (known as quaternary ammonium compounds (QACs)) are typically cations. According to the methods and compositions disclosed herein, any compound that contains a quaternary nitrogen atom or a nitrogen atom that can be converted into a quaternary nitrogen atom (cation precursor) can be a suitable cation for the disclosed ionic liquids.

In some examples, phosphorous atoms can exist as a charged phosphonium species, for example, through alkylation of the phosphorous atom. Thus, compounds that possess a quaternary phosphorous atom (known as quaternary phosphonium compounds) are typically cations. According to the methods and compositions disclosed herein, any compound that contains a quaternary phosphorus atom or a phosphorus atom that can be converted into a quaternary phosphonium atom can be a suitable cation for the disclosed ionic liquids.

In some examples, sulfur atoms can exist as a charged sulfonium species, for example, through alkylation of the sulfurous atom. Thus, compounds that possess a ternary sulfurous atom are typically cations. According to the methods and compositions disclosed herein, any compound that contains a ternary sulfurous atom or a sulfurous atom that can be converted into a ternary sulfurous atom can be a suitable cation for the disclosed ionic liquids.

Some specific organic cations suitable for use herein are heteroaryls. In some embodiments, the heteroaryl can be an aliphatic heteroaryl. An aliphatic heteroaryl cation is a compound that comprises at least one aliphatic moiety bonded to a heteroaryl moiety. In the aliphatic heteroaryl cation, the aliphatic moiety can be any alkyl, alkenyl, alkynyl, heteroalkyl, heteroalkenyl, heteroalkynyl, cycloalkyl, cycloalkenyl, heterocycloalkyl, or heterocycloalkenyl group, as described herein.

In the heteroaryl cation, the heteroaryl moiety can be any heteroaryl moiety as described herein. For example, the heteroaryl moiety can be an aryl group having a nitrogen atom and optionally one or more heteroatoms (e.g., oxygen, sulfur, phosphorous, or halonium). Examples of specific heteroaryl moieties that can be used in the heteroaryl cations include, but are not limited to, substituted or unsubstituted benztriazoliums, substituted or unsubstituted benzimidazoliums, substituted or unsubstituted benzothiazoliums, substituted or unsubstituted pyridiniums, substituted or unsubstituted pyridaziniums, substituted or unsubstituted pyrinidiniums, substituted or unsubstituted pyraziniums, substituted or unsubstituted imidazoliums, substituted or unsubstituted pyrazoliums, substituted or unsubstituted oxazoliums, substituted or unsubstituted 1,2,3-triazoliums, substituted or unsubstituted 1,2,4-triazoliums, substituted or unsubstituted thiazoliums, substituted or unsubstituted piperidiniums, substituted or unsubstituted pyrrolidiniums, substituted or unsubstituted quinoliums, and substituted or unsubstituted isoquinoliums. As described herein, when the heteroatom of the heteroaryl is nitrogen, this forms a quaternary ammonium cation.

Some specific organic cations suitable for use herein are cyclic compounds comprising one or more heteroatoms. For example, the organic cation can comprise a pyridinyl moiety, imidazolinyl moiety, or the like that can have substituted or unsubstituted linear or branched alkyl units attached thereto. In some examples, the organic cation can comprise a single heteroatom wherein a sufficient number of substituted or unsubstituted linear or branched alkyl units are attached to the heteroatom such that a cation is formed. For example, the organic cation can comprise Cₙ alkylmethylimidazolium [Cₙmim] where n is an integer of from 1 to 8. Preferably, the cation C₁₋₄ alkyl-methylimidazolium [C₁₋₄mim] can be used. Other non-limiting examples of heterocyclic and heteroaryl units that can be alkylated to form cationic units include substituted or unsubstituted furans, substituted or unsubstituted benzofurans, substituted or unsubstituted dibenzofurans, substituted or unsubstituted indolizines, substituted or unsubstituted isoindoles, substituted or unsubstituted indoles, substituted or unsubstituted indolines, substituted or unsubstituted indazoles, substituted or unsubstituted imidazoles, substituted or unsubstituted morpholiniums, substituted or unsubstituted morpholines, substituted or unsubstituted oxazoles, substituted or unsubstituted oxaphospholes, substituted or unsubstituted oxothiazoles, substituted or unsubstituted oxazines, substituted or unsubstituted oxazolines, substituted or unsubstituted phenazine, substituted or unsubstituted phthalazines, substituted or unsubstituted purines, substituted or unsubstituted pyrroles, substituted or unsubstituted pyrazoles, substituted or unsubstituted pyridines, substituted or unsubstituted pyrazines, substituted or unsubstituted pyrimidines, substituted or unsubstituted pryidazines, substituted or unsubstituted phospholes, substituted or unsubstituted pentazoles, substituted or unsubstituted pyridazines, substituted or unsubstituted piperazines, substituted or unsubstituted piperidines, substituted or unsubstituted pyrans, substituted or unsubstituted isoquinolines, substituted or unsubstituted quinolines, substituted or unsubstituted quinoxalines, substituted or unsubstituted quinazolines, substituted or unsubstituted selenozoles, substituted or unsubstituted triazoles, substituted or unsubstituted thiazoles, substituted or unsubstituted isothiazoles, substituted or unsubstituted dithiazoles, substituted or unsubstituted azathiazoles, substituted or unsubstituted thiophenes, substituted or unsubstituted benzothiophenes, substituted or unsubstituted dibenzothiophenes, substituted or unsubstituted tetrazoles, substituted or unsubstituted thiadiazoles, and the like, including derivatives and mixtures thereof.

In some examples, the disclosed ionic liquid can comprise an ammonium cation of the structure NR¹R²R³R⁴, wherein R¹, R², R³, and R⁴ are independently H, halogen, substituted or unsubstituted C₁-C₈ alkyl, substituted or unsubstituted C₁-C₈ cycloalkyl.

In some examples, R¹, R², R³, and R⁴ are independently H or substituted or unsubstituted C₁-C₈ alkyl. In some examples, R¹, R², R³, and R⁴ are independently H or unsubstituted C₁-C₈ alkyl. In some examples, R¹, R², R³, and R⁴ are independently H or unsubstituted C₁-C₄ alkyl. In some examples, R¹ is H. In some examples, R², R³ and R⁴ are C₂H₅. In some examples, the at least one ammonium cation comprises [HN(C₂H₅)₃]⁺.

In some examples, the ionic liquid can comprise [HN₂₂₂][Al₂Cl₇], where the notation “HN₂₂₂” indicates a triethylammonium group.

In some examples, the disclosed ionic liquid compositions can comprise a phosphonium cation of the structure PR¹R²R³R⁴, wherein R¹, R², R³, and R⁴ are independently H, halogen, substituted or unsubstituted C₁-C₂₀ alkyl, substituted or unsubstituted C₁-C₈ cycloalkyl, or wherein, as valence permits, two or more of R¹, R², R³, and R⁴, together with the atoms to which they are attached, form a 3-10 membered cyclic moiety.

Magnetic Particles

The magnetic particle can be any magnetic particle that can be conjugated to the ionic liquid and which can provide for localization of the bound ionic liquid under an applied magnetic field.

The magnetic particle can be of any shape (e.g., a sphere, a rod, a quadrilateral, an ellipse, a triangle, a polygon, etc.). In some examples, the shape of the magnetic particle can be selected to facilitate the conjugation of the ionic liquid. The magnetic particle can comprise any suitable material, for example iron, cobalt, zinc, nickel, manganese, silver, gold, cadmium, or a combination thereof. In some examples, the magnetic particle can comprise any suitable metal compound, such as an oxide of iron, cobalt, zinc, nickel, manganese, or a combination thereof. In some examples, the magnetic particle can comprise an iron oxide, for example Fe₃O₄. In some examples, the plurality of magnetic particles are superparamagnetic.

The magnetic particle can have an average particle size. “Average particle size” and “mean particle size” are used interchangeably herein, and generally refer to the statistical mean particle size of the particles in a population of particles. For a particle with a substantially spherical shape, the diameter of a particle can refer, for example, to the hydrodynamic diameter. As used herein, the hydrodynamic diameter of a particle can refer to the largest linear distance between two points on the surface of the particle. Mean particle size can be measured using methods known in the art, such as evaluation by scanning electron microscopy, transmission electron microscopy, and/or dynamic light scattering.

The magnetic particles can, for example, have an average particle size of 10 nm or more (e.g., 15 nm or more, 20 nm or more, 25 nm or more, 30 nm or more, 35 nm or more, 40 nm or more, 45 nm or more, 50 nm or more, 55 nm or more, 60 nm or more, 65 nm or more, 70 nm or more, 75 nm or more, 80 nm or more, 85 nm or more, 90 nm or more, 95 nm or more, 100 nm or more, 110 nm or more, 120 nm or more, 130 nm or more, 140 nm or more, 150 nm or more, 160 nm or more, 170 nm or more, 180 nm or more, 190 nm or more, 200 nm or more, 225 nm or more, 250 nm or more, 275 nm or more, 300 nm or more, 325 nm or more, 350 nm or more, 400 nm or more, 450 nm or more, 500 nm or more, 550 nm or more, 600 nm or more, 700 nm or more, or 800 nm or more). In some examples, the magnetic particle can have an average particle size of 1 micrometer (μm, micron) or less (e.g., 900 nm or less, 800 nm or less, 700 nm or less, 600 nm or less, 550 nm or less, 500 nm or less, 450 nm or less, 400 nm or less, 350 nm or less, 325 nm or less, 300 nm or less, 275 nm or less, 250 nm or less, 225 nm or less, 200 nm or less, 190 nm or less, 180 nm or less, 170 nm or less, 160 nm or less, 150 nm or less, 140 nm or less, 130 nm or less, 120 nm or less, 110 nm or less, 100 nm or less, 95 nm or less, 90 nm or less, 85 nm or less, 80 nm or less, 75 nm or less, 70 nm or less, 65 nm or less, 60 nm or less, 55 nm or less, 50 nm or less, 45 nm or less, 40 nm or less, 35 nm or less, 30 nm or less, or 25 nm or less). The average particle size of the magnetic particle can range from any of the minimum values described above to any of the maximum values described above. For example, the magnetic particle can have an average particle size of from 10 nm to 1 μm (e.g., from 10 nm to 800 nm, from 10 nm to 600 nm, from 10 nm to 500 nm, from 20 nm to 400 nm, from 25 nm to 350 nm, from 30 nm to 300 nm, from 35 nm to 250 nm, from 40 nm to 200 nm, from 40 nm to 150 nm, from 40 nm to 100 nm, from 40 nm to 60 nm, or from 45 nm to 55 nm).

In some examples, the magnetic particles can be substantially monodisperse. “Monodisperse” and “homogeneous size distribution,” as used herein, and generally describe a population of particles where all of the particles are the same or nearly the same size. As used herein, a monodisperse distribution refers to particle distributions in which 80% of the distribution (e.g., 85% of the distribution, 90% of the distribution, or 95% of the distribution) lies within 25% of the mean particle size (e.g., within 20% of the mean particle size, within 15% of the mean particle size, within 10% of the mean particle size, or within 5% of the mean particle size).

Methods of Making

Also disclosed herein are methods of making the magnetic particle-ionic liquid composite materials disclosed herein. For example, the methods of making the magnetic particle-ionic liquid composite materials can comprise contacting the magnetic particle with the ionic liquid. In some examples, the method is performed under an inert atmosphere, such as an argon atmosphere.

In the disclosed methods, the magnetic particle need not be coated before it is contacted with the ionic liquid. For example, the magnetic particle is not coated with silica or a silane before being contacted with the ionic liquid.

Methods of Use

The magnetic particle-ionic liquid composite materials can, for example, be used as catalysts for Lewis acid catalysis. For example, also disclosed herein are methods of alkylating an aryl substrate comprising combining an aryl substrate with an alkylating agent in the presence of a catalyst, thereby alkylating the aryl substrate and forming a mixture, wherein the catalyst comprises any of the magnetic particle-ionic liquid composite materials disclosed herein.

The ionic liquids disclosed herein can be used in the disclosed methods neat; that is, there are no or substantially no (e.g., less than 5, 4, 3, 2, or 1 mole %) solvents or other materials besides the aryl substrate, the alkylating agent, and the magnetic particle-ionic liquid composite material present in the reaction. For example, the disclosed methods can be solventless or substantially solventless wherein there is no solvent or other materials in the reaction besides the reactants and magnetic particle-ionic liquid composite material.

In some examples, the aryl substrate can comprise a compound of formula I:

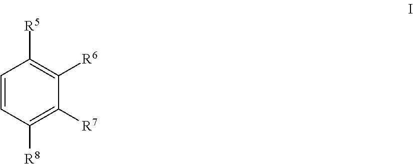

wherein R⁵, R⁶, R⁷, and R⁸ are independently H, halogen, hydroxyl, a substituted or unsubstituted C₁-C₂₀ alkyl, substituted or unsubstituted C₂-C₂₀ alkenyl, substituted or unsubstituted C₂-C₂₀ alkynyl, substituted or unsubstituted C₄-C₂₀ alkylaryl, substituted or unsubstituted C₄-C₂₀ alkylcycloalkyl, or wherein, as valence permits, two or more of R⁵, R⁶, R⁷, and R⁸, together with the atoms to which they are attached, form a 3-10 membered (poly)cyclic moiety. As used herein “(poly)cyclic” includes both cyclic moieties and poly cyclic moieties, wherein a polycyclic moiety includes 2 or more cyclic moieties.

In some examples, R⁵, R⁶, R⁷, and R⁸ are independently H, a substituted or unsubstituted C₁-C₂₀ alkyl, or wherein, as valence permits, two or more of R⁵, R⁶, R⁷, and R⁸, together with the atoms to which they are attached, form a 3-10 membered (poly)cyclic moiety. In some examples, the aryl substrate can comprise benzene, xylene, or anthracene.

The alkylating agent can, for example, comprise a compound of formula II:

R⁹X II

wherein X is a halogen; and R⁹ is a substituted or unsubstituted C₁-C₂₀ alkyl, substituted or unsubstituted C₂-C₂₀ alkenyl, substituted or unsubstituted C₂-C₂₀ alkynyl, substituted or unsubstituted C₄-C₂₀ alkyl aryl, or substituted or unsubstituted C₄-C₂₀ alkylcycloalkyl.

In some examples, X is chlorine. In some examples, R⁹ is a substituted or unsubstituted C₄-C₂₀ alkylaryl. In some examples, R⁹ is a substituted C₄-C₂₀ alkylaryl. In some examples, R⁹ is a substituted benzyl. In some examples, the alkylating agent comprises benzyl chloride.

The catalyst can, for example, be provided in an amount of 1.2 mol % or more of ionic liquid loading relative to the amount of the aryl substrate (e.g., 1.3 mol % or more, 1.4 mol % or more, 1.5 mol % or more, 1.6 mol % or more, 1.7 mol % or more, or 1.8 mol % or more). In some examples, the catalyst can be provided in an amount of 2 mol % or less of ionic liquid loading relative to the amount of the aryl substrate (e.g., 1.9 mol % or less, 1.8 mol % or less, 1.7 mol % or less, 1.6 mol % or less, 1.5 mol % or less, or 1.4 mol % or less). The amount of catalyst provided can range from any of the minimum values described above to any of the maximum values described above. For example, the catalyst can be provided in an amount of from 1.2 mol % to 2 mol % of ionic liquid loading relative to the amount of the aryl substrate (e.g., from 1.2 mol % to 1.6 mol %, from 1.6 mol % to 2 mol %, from 1.2 mol % to 1.4 mol %, from 1.4 mol % to 1.6 mol %, from 1.6 mol % to 1.8 mol %, from 1.8 mol % to 2 mol %, or from 1.3 mol % to 1.9 mol %).

In some examples, the alkylated aryl substrate is produced in a yield of 100%. In some examples, the alkylated aryl substrate can be produced with a selectivity of 60% or more (e.g., 61% or more, 62% or more, 63% or more, 64% or more, 65% or more, 66% or more, 67% or more, 68% or more, 69% or more, 70% or more, 75% or more, 80% or more, 85% or more, 90% or more, or 95% or more).

In some examples, the method is performed at room temperature. As used herein, room temperature is meant to include temperatures of 20-30° C.

The methods can further comprise separating the catalyst from the mixture, thereby forming a recycled catalyst. In some examples, the recycled catalyst is used to contact the aryl substrate and the alkylating agent. In some examples, the catalyst can be recycled 2 or more times (e.g., 3 or more, 4 or more, 5 or more, 6 or more, 7 or more, or 8 or more).

Separating the catalyst from the mixture can, for example, comprise decanting, centrifugation, filtration, or a combination thereof. In some examples, separating the catalyst from the mixture can comprise magnetically separating the catalyst from the mixture. Magnetically separating the catalyst from the mixture can, for example, comprise applying a magnetic field to the mixture. The magnetic field can be generated by any means consistent with the methods described herein, for example by a permanent magnet, an electromagnet, or a combination thereof.

### EXAMPLES

The following examples are set forth below to illustrate the methods and results according to the disclosed subject matter. These examples are not intended to be inclusive of all aspects of the subject matter disclosed herein, but rather to illustrate representative methods and results. These examples are not intended to exclude equivalents and variations of the present invention, which are apparent to one skilled in the art.

Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, temperatures, pressures and other reaction ranges and conditions that can be used to optimize the product purity and yield obtained from the described process. Only reasonable and routine experimentation will be required to optimize such process conditions.

### Example 1

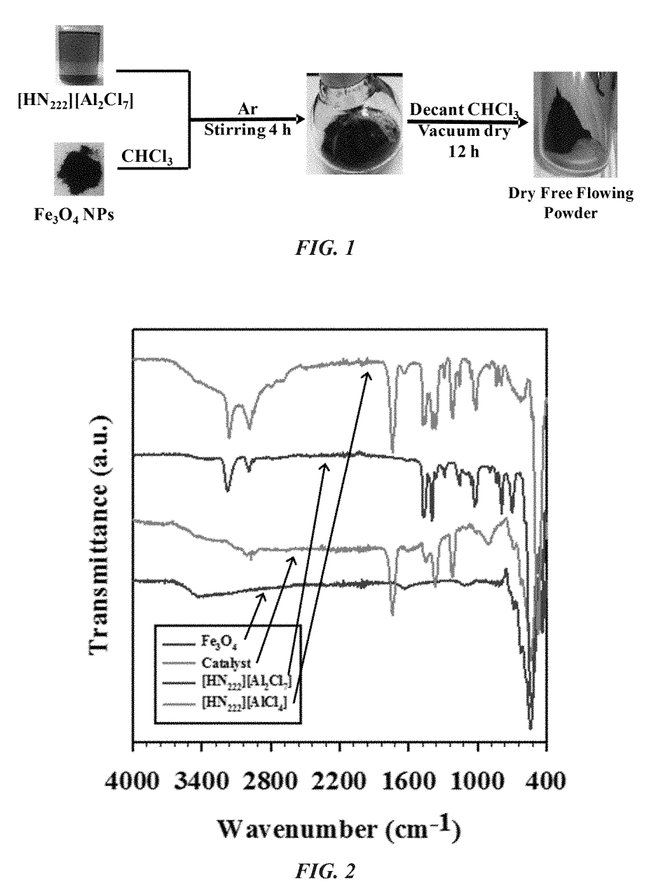

chloroaluminate ionic liquid supported Fe₃O₄ catalysts were prepared by loading 10 to 40 wt % of the ionic liquid on a nanopowder (40-50 nm) of Fe₃O₄ nanoparticles (NPs), as shown below in Scheme 1. When 10 wt %, 20 wt %, and 30, wt % of [HN₂₂₂][Al₂Cl₇] ionic liquid (IL) was added to the suspension of Fe₃O₄ nanoparticles in chloroform, a heterogeneous system with a viscus black solid at the bottom and a brown liquid formed (FIG. 1). After stirring for 4 h, the chloroform layer was decanted and the solid remained at the bottom. The sticky solid was dried under vacuum for 12 h and resulting in free flowing solid catalyst. When 10-30 wt % of ionic liquid loaded on the Fe₃O₄ nanoparticles, a free flowing solid was observed. However, when 40 wt % of ionic liquid was loaded on the Fe₃O₄ nanoparticles, a sticky solid was obtained. By following a similar procedure, [HN₂₂₂][Al₂Cl₇] was loaded on conventional Fe₃O₄ and Fe₂O₃ nanoparticles.

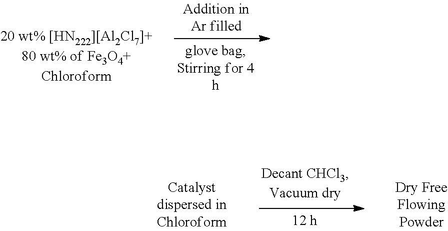

### Scheme 1. Synthesis of Magnetic Particle-Ionic Liquid Composite Materials.

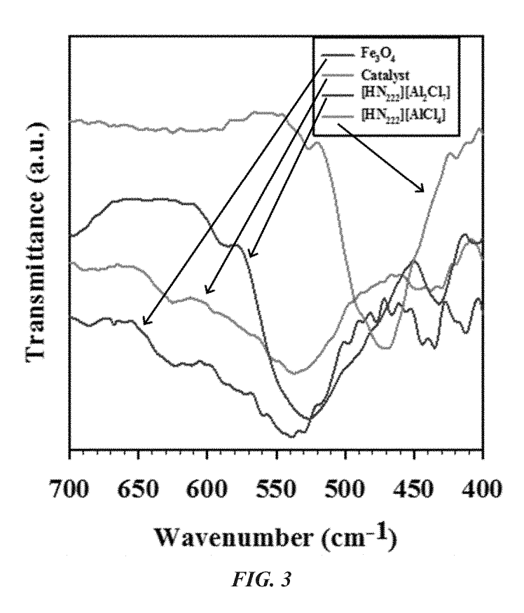

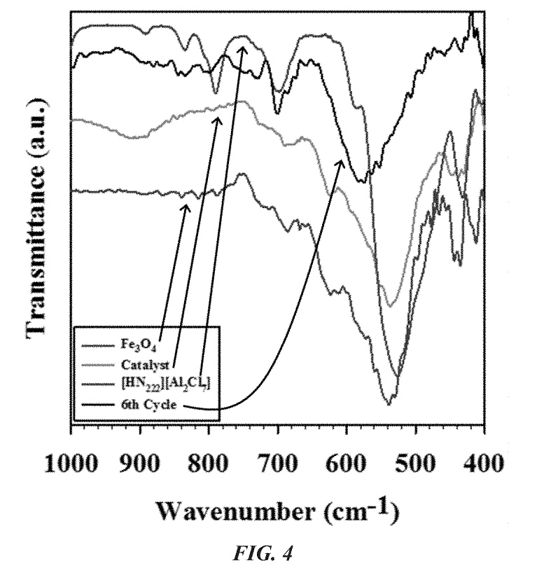

To see the presence of the ionic liquid on the solid support, infrared (IR) analysis of 20 wt % of ionic liquid loaded on Fe₃O₄ was carried out and compared with Fe₃O₄, [HN₂₂₂][Al₂Cl₇], and [HN₂₂₂][AlCl₄] (FIG. 2-FIG. 4). The IR band of [HN₂₂₂][Al₂Cl₇] appears in the IR spectrum of the catalyst but the peak is shifted, which indicates that ionic liquid is present in the catalyst. For chloroaluminate ionic liquids, a peculiar peak for Al—Cl stretch appears in the region 600-450 cm⁻¹ (Mains G J et al. J. Phys. Chem. A 2001, 105, 4371-4378). For the catalyst, a peak at 535 cm⁻¹ appears, which is lying in between the Al—Cl stretch peak of an [Al₂Cl₇]⁻ anion (525 cm⁻¹) and the Fe—O peak of γ-Fe₂O₃ (540 cm⁻¹) (Mohapatra J et al. RSC Advances 2015, 5, 14311-14321). The peak for Al—Cl stretch for [AlCl₄]⁻ anion appears at 470 cm⁻¹. This indicates that the ionic liquid loaded on Fe₃O₄ contains [Al₂Cl₇]⁻ anion. In the catalyst, a new peak appeared at 906 cm⁻¹ and can be due to interaction of the ionic liquid with the solid Fe₃O₄ nanoparticles. The IR spectrum of 10 wt %, 20 wt %, and 30 wt % loaded ionic liquid on Fe₃O₄ nanoparticles was same. Analysis of the recycled catalyst after a 6^{th} run indicated diminishing of the peak at 535 cm⁻¹, which can indicate leaching of Al from catalyst after 6^{th} run.

TABLE 1
Observations in preparation of [HN₂₂₂][Al₂Cl₇] ionic liquid
(IL) loaded iron oxide catalyst.

| | | | | Physical Observations | | |
|---|---|---|---|---|---|---|
| | | IL | | | | After |
| S. | | Loading | | | | vacuum |
| N. | Support | (wt %) | Solvent | After addition | After 4 h stirring | drying |
| 1 | Fe₃O₄ | 20 | Neat | Heterogeneous sticky and powder mass | | |
| | NPs | | | | | |
| 2 | Fe₃O₄ | 10 | Chloroform | Heterogeneous system with suspended solid | | Dark grey |
| | NPs | | | in light grey colored liquid | | free |
| 3 | Fe₃O₄ | 20 | | | | flowing |
| | NPs | | | | | powder |
| 4 | Fe₃O₄ | 30 | | | | Faint |
| | NPs | | | | | brown free |
| | | | | | | flowing |
| | | | | | | powder |
| 5 | Fe₃O₄ | 40 | | Heterogeneous | Sticky mass in light | Sticky |
| | NPs | | | system with | grey liquid | mass |
| | | | | suspended solid in | | |
| | | | | light grey colored | | |
| | | | | liquid | | |
| 6 | Fe₃O₄ | 20 | | Heterogeneous | Heterogeneous | Brown |
| | | | | system with | system with | free |
| | | | | suspended solid in | suspended solid in | flowing |
| | | | | colorless liquid | light brown colored | powder |
| | | | | | liquid | |
| 7 | Fe₂O₃ | 20 | | Heterogeneous | Heterogeneous | Brown |
| | NPs | | | system with | system with | free |
| | | | | suspended solid in | suspended solid in | flowing |
| | | | | colorless liquid | light brown colored | powder |
| | | | | | liquid | |

Reaction conditions: Fe₃O₄ nanoparticles (NPs) suspended in 3 ml CHCl₃, [HN₂₂₂][Al₂Cl₇], Stir 4 h, decant solvent and vacuum dry for 12 h.

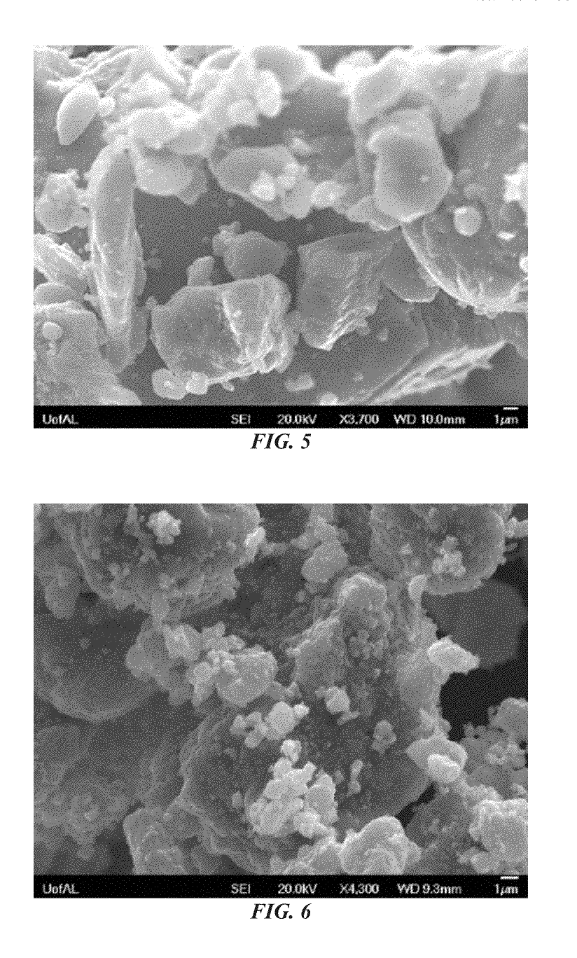

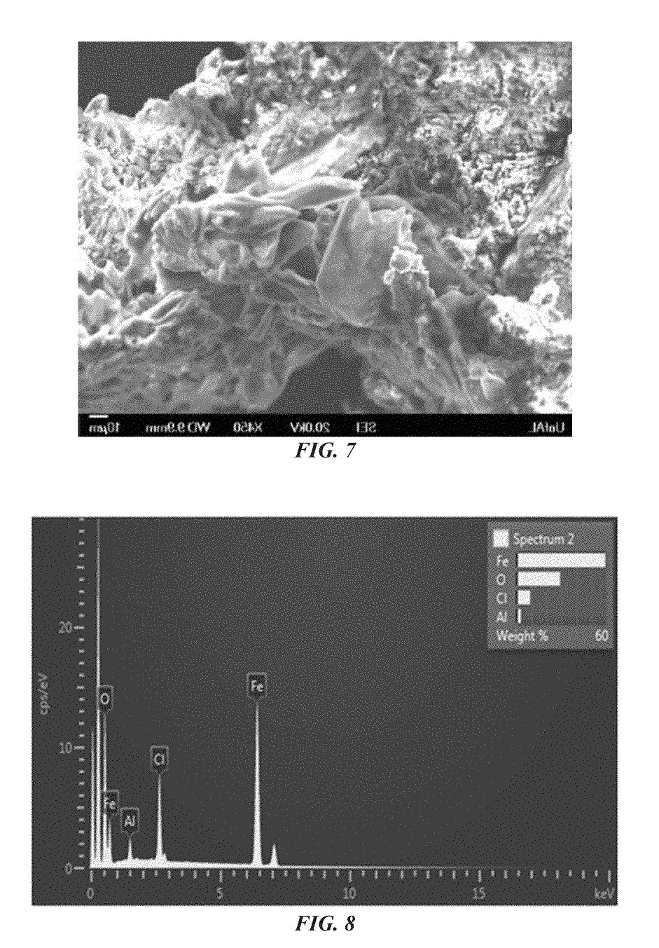

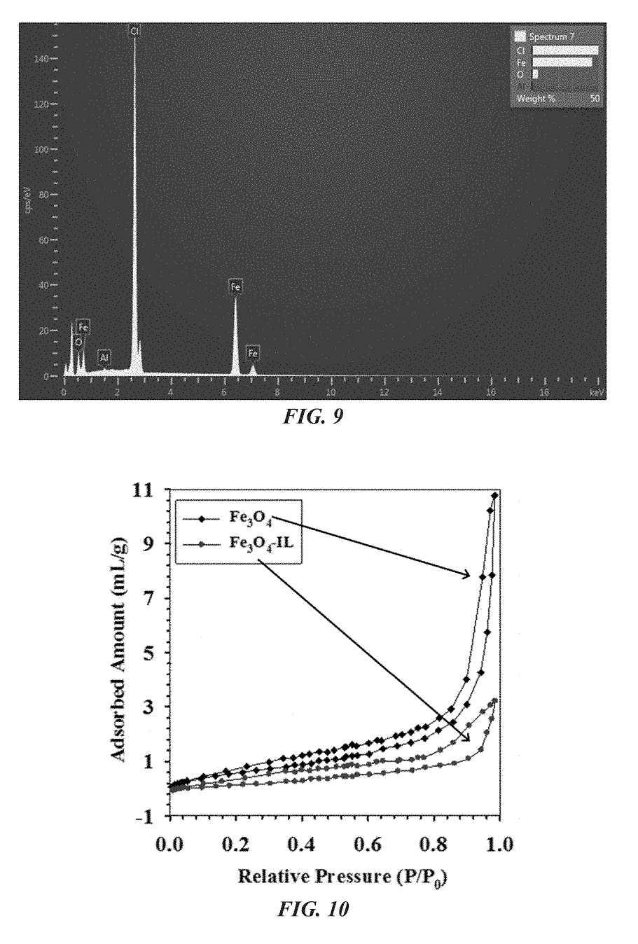

To check the ionic liquid loading and the surface morphology of the catalyst, SEM-EDX analysis of 20 wt % of ionic liquid loaded on Fe₃O₄ catalyst was carried out. The ionic liquid is loaded on to a solid support (FIG. 5 and FIG. 6), and the SEM image of a recycled catalyst indicates agglomeration of nanoparticles (FIG. 7). In EDX analysis (FIG. 8-FIG. 9), it was found that in 20 wt % ionic liquid loaded Fe₃O₄ nanoparticles, the Al content was 2.74 wt % (Table 2), which matched the Al content in the ionic liquid during catalyst preparation. This confirmed that all ionic liquid used in catalyst preparation was loaded on the solid support. In EDX analysis of recycled catalysts, the Al content decreased from 2.74 to 0.54 wt % after 6 cycles, indicating that there was leaching of Al over the six runs. EDX analysis of the recycled catalysts further shows an increase in Cl content, which can be assigned to HCl released during the alkylation reaction.

TABLE 2
Elemental composition of fresh and recycled catalyst
determined by EDX analysis.

Elemental composition (wt %)

| Elements | Fresh catalyst | After 6^{th} cycle |
|---|---|---|
| Fe | 59.35 | 47.50 |
| O | 28.98 | 7.92 |
| Al | 2.73 | 0.54 |
| Cl | 8.94 | 44.04 |

The textural properties of the 20 wt % of ionic liquid loaded Fe₃O₄ nanoparticles and Fe₃O₄ nanoparticles were investigated by N₂-adsorption (FIG. 10). After ionic liquid loading, the surface area of the solid support decreased from 38.4 to 13.6 m²/g (FIG. 10). The results also indicated that the solid support material, Fe₃O₄ nanoparticles, was non-porous (FIG. 10).

To test the catalytic activity of prepared catalyst, alkylation of benzene with benzyl chloride as an Lewis acid catalyzed reaction was investigated. Catalytic activity was tested for 10 wt %, 20 wt %, and 30 wt % [HN₂₂₂][Al₂Cl₇] ionic liquid loaded Fe₃O₄ nanoparticles (Fe(II), Fe(III)) at optimized reaction conditions and results are summarized in Table 3. The 20 wt % ionic liquid loaded Fe₃O₄ nanoparticles gave 100% conversion with 77% product selectivity of the desired diphenyl methane product. The 30 wt % ionic liquid loaded Fe₃O₄ nanoparticles gave 100% conversion with a moderate 61% product selectivity, which can be due to the presence of excess of ionic liquid. No activity was observed for 10 wt % ionic liquid loaded Fe₃O₄ nanoparticles.

As in magnetite, iron exists in +2 and +3 oxidation states. A control experiment using Fe₂O₃ nanoparticles (Fe(III)) as a solid support was carried out to identify the reason behind the inactivity of the 10 wt % ionic liquid loaded Fe₃O₄ nanoparticles. When 20 wt % [HN₂₂₂][Al₂Cl₇] ionic liquid was loaded on Fe₂O₃ nanoparticles, no activity was observed. This indicated that Fe₂O₃ is deactivating the ionic liquid, and can be attributed to the fewer number of coordinatively unsaturated metal sites (CUS) in Fe₂O₃ as compared to Fe₃O₄ (Yang F et al. Natl. Sci. Rev. 2015, 2, 183-201). This reason for inactivity can also be assigned to the lack of activity in the 10 wt % [HN₂₂₂][Al₂Cl₇] ionic liquid loaded Fe₃O₄ nanoparticles.

No catalytic activity was observed with neat [HN₂₂₂][AlCl₄] and neat Fe₃O₄ nanoparticles. At the investigated reaction conditions, the product selectivity using 20 wt % ionic liquid loaded Fe₃O₄ nanoparticles was higher than the product selectivity for conventional [HN₂₂₂][Al₂Cl₇] ionic liquid and AlCl₃.

TABLE 3
Comparative catalytic activity of catalysts with different solid supports.

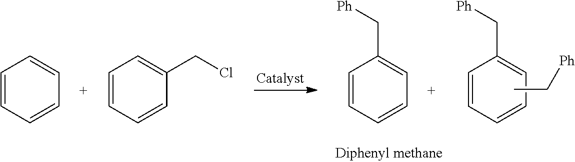

| | | | Activity | |
|---|---|---|---|---|
| | | Time | Conv. | Diphenyl methane |
| S.N. | Catalyst | (h) | (%) | Sel. (%) |
| 1 | [HN₂₂₂][Al₂Cl₇] | 0.5 | 100 | 65 |
| 2 | [HN₂₂₂][AlCl₄] | 0.5 | 0 | 0 |
| 3 | AlCl₃ | 0.5 | 100 | 50 |
| 4 | Fe₃O₄ | 0.5 | 0 | 0 |
| 5 | 10 wt % [HN₂₂₂][Al₂Cl₇]—Fe₃O₄ NPs | 4 | 0 | 0 |
| 6 | 20 wt % [HN₂₂₂][Al₂Cl₇]—Fe₃O₄ NPs | 0.5 | 100 | 77 |
| 7 | 30 wt % [HN₂₂₂][Al₂Cl₇]—Fe₃O₄ NPs | 0.5 | 100 | 61 |
| 8 | 20 wt % [HN₂₂₂][Al₂Cl₇]—Fe₂O₃ NPs | 4 | 0 | 0 |
| 9 | 20 wt % [HN₂₂₂][Al₂Cl₇]—Fe₃O₄ | 0.5 | 0 | 0 |
| 10 | 20 wt % [HN₂₂₂][Al₂Cl₇]—Fe₃O₄ | 4 | 100 | 46 |

Reaction conditions: Catalyst (1.2 mol % ionic liquid loading), benzyl chloride (1 mmol), benzene (10 mmol) stirred at room temperature.

To check the substrate scope, the best 20 wt % ionic liquid loaded Fe₃O₄ nanoparticles was investigated in alkylation of aromatic compounds such as toluene, o-, m-, p-xylene and results are summarized in Table 4. The highest monoalkylated product selectivity was observed in alkylation of p-xylene.

TABLE 4
Substrate scope.

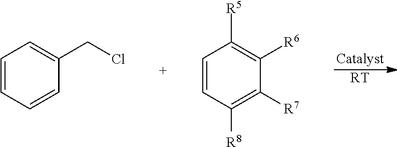

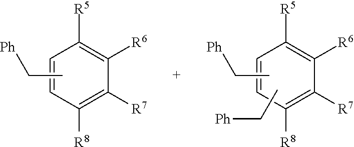

| | | Activity | |
|---|---|---|---|
| | | Conv. | Monoalkylation Sel. |
| S.N. | Substrate | (%) | (%) |
| 1 | R⁵ = CH₃, R⁶-R⁸ = H | 100 | 44 |
| 2 | R⁵, R⁶ = CH₃; R⁷, R⁸ = H | 100 | 44 |
| 3 | R⁵, R⁷ = CH₃; R⁶, R⁸ = H | 100 | 54 |
| 4 | R⁵, R⁸ = CH₃, R⁶, R⁷ = H | 100 | 61 |

Reaction conditions: 20 wt % IL loaded Fe₃O₄ NPs (1.2 mol % IL loading), benzyl chloride (1 mmol), substrate (1 mmol) stirred at RT for 0.5 h.

To test the recyclability of the [HN₂₂₂][Al₂Cl₇] ionic liquid loaded Fe₃O₄ nanoparticles, after completion of a reaction the catalyst was held with an external magnet and the reaction mixture was taken out using a syringe. The catalyst was washed with hexane three times and dried under high vacuum. This catalyst was then used for the next catalytic run. The catalyst was used for 5 runs without any loss of catalytic activity, with decreased activity being observed in the 6^{th} run (Table 5). For comparison, [HN₂₂₂][Al₂Cl₇] ionic liquid was recycled and after the first run, the upper reaction mixture of the biphasic system was collected with a syringe and the remaining ionic liquid at the bottom was washed with hexane 3 times. The ionic liquid was dried under high vacuum and used to catalyze the next run. When the ionic liquid alone was recycled, the catalytic activity reduced after the 1^{st} run (Table 5).

TABLE 5
Recyclability of 20 wt % ionic liquid (IL) loaded
Fe₃O₄ nanoparticles (NPs).

| | |
|---|---|
| Activity of 20 wt % | Activity of |
| IL loaded Fe₃O₄ | [HN₂₂₂][Al₂Cl₇] |

| Run | Conv. (%) | Sel. (%) | Conv. (%) | Sel. (%) |
|---|---|---|---|---|
| 1 | 100 | 76.5 | 100 | 65.5 |
| 2 | 100 | 73.32 | 15 | 100 |
| 3 | 100 | 70.47 | | |
| 4 | 100 | 70.25 | | |
| 5 | 100 | 67.21 | | |
| 6 | 73 | 67 | | |

Reaction conditions: Catalyst (1.2 mol % ionic liquid loading), benzyl chloride (1 mmol), benzene (10 mmol) stirred at room temperature for 0.5 h.

The methods and compositions of the appended claims are not limited in scope by the specific methods and compositions described herein, which are intended as illustrations of a few aspects of the claims and any methods and compositions that are functionally equivalent are within the scope of this disclosure. Various modifications of the methods and compositions in addition to those shown and described herein are intended to fall within the scope of the appended claims. Further, while only certain representative methods, compositions, and aspects of these methods and compositions are specifically described, other methods and compositions and combinations of various features of the methods and compositions are intended to fall within the scope of the appended claims, even if not specifically recited. Thus, a combination of steps, elements, components, or constituents can be explicitly mentioned herein; however, all other combinations of steps, elements, components, and constituents are included, even though not explicitly stated.

What is claimed is:

1. A magnetic particle-ionic liquid composite material, comprising:
an ionic liquid conjugated to a magnetic particle;
wherein the ionic liquid comprises at least one cation and at least one metal halide anion, wherein the ionic liquid is a salt of the at least one cation and the at least one metal halide anion with a melting point of 150° C. or less; and
wherein the ionic liquid is not covalently bound to the magnetic particle.

2. The composite material of claim 1, wherein the at least one cation is an ammonium cation, an imidazolium cation, a pyridinium cation, a phosphonium cation, a sulphonium cation, or a combination thereof.

3. The composite material of claim 1, wherein the at least one cation comprises an ammonium cation of the structure NR¹R²R³R⁴, wherein R¹, R², R³, and R⁴ are independently H, halogen, substituted or unsubstituted C₁-C₈ alkyl, or substituted or unsubstituted C₁-C₈ cycloalkyl.

4. The composite material of claim 3, wherein R¹, R², R³, and R⁴ are independently H or substituted or unsubstituted C₁-C₈ alkyl.

5. The composite material of claim 3, wherein the at least one ammonium cation comprises [HN(C₂H₅)₃]⁺.

6. The composite material of claim 1, wherein the at least one cation comprises a phosphonium cation of the structure PR¹R²R³R⁴, wherein R¹, R², R³, and R⁴ are independently H, halogen, substituted or unsubstituted C₁-C₂₀ alkyl, substituted or unsubstituted C₁-C₈ cycloalkyl, or wherein, as valence permits, two or more of R¹, R², R³, and R⁴, together with the atoms to which they are attached, form a 3-10 membered cyclic moiety.

7. The composite material of claim 1, wherein the at least one metal halide anion comprises a metal chloride, an aluminum halide, or a combination thereof.

8. The composite material of claim 1, wherein the at least one metal halide anion comprises a chloroaluminate.

9. The composite material of claim 1, wherein the at least one metal halide anion comprises [Al₂Cl₁]⁻.

10. The composite material of claim 1, wherein the magnetic particle has an average particle size of from 10 nm to 1 μm.

11. The composite material of claim 1, wherein the magnetic particle-ionic liquid composite material comprises from 1 wt % to 35 wt % of the ionic liquid, based on the total weight of the magnetic particle-ionic liquid composite material.

12. The composite material of claim 1, wherein the at least one cation of the ionic liquid is not covalently bound to the magnetic particle.

13. A method of alkylating an aryl substrate comprising combining an aryl substrate with an alkylating agent in the presence of a catalyst, thereby alkylating the aryl substrate and forming a mixture, wherein the catalyst comprises the composite material of claim 1.

14. The method of claim 13, wherein the aryl substrate comprises a compound of formula I: 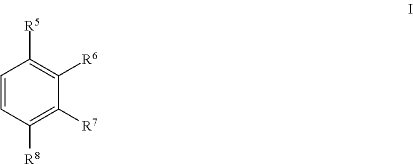
wherein R⁵, R⁶, R⁷, and R⁸ are independently H, halogen, hydroxyl, a substituted or unsubstituted C₁-C₂₀ alkyl, substituted or unsubstituted C₂-C₂₀ alkenyl, substituted or unsubstituted C₂-C₂₀ alkynyl, substituted or unsubstituted C₄-C₂₀ alkylaryl, substituted or unsubstituted C₄-C₂₀ alkylcycloalkyl, or wherein, as valence permits, two or more of R⁵, R⁶, R⁷, and R⁸, together with the atoms to which they are attached, form a 3-10 membered (poly)cyclic moiety.

15. The method of claim 13, wherein the alkylating agent comprises a compound of formula II:
R⁹X II
wherein
X is a halogen; and
R⁹ is a substituted or unsubstituted C₁-C₂₀ alkyl, substituted or unsubstituted C₂-C₂₀ alkenyl, substituted or unsubstituted C₂-C₂₀ alkynyl, substituted or unsubstituted C₄-C₂₀ alkylaryl, or substituted or unsubstituted C₄-C₂₀ alkylcycloalkyl.

16. The method of claim 13, wherein the catalyst is provided in an amount of from 1.2 mol % to 2 mol % of ionic liquid loading relative to the amount of the aryl substrate.

17. The method of claim 13, wherein the alkylated aryl substrate is produced with a selectivity of 60% or more.

18. The method of claim 13, further comprising separating the catalyst from the mixture, thereby forming a recycled catalyst.

19. The method of claim 18, wherein the recycled catalyst is used to contact the aryl substrate and the alkylating agent.

20. The method of claim 18, wherein the catalyst is recycled 2 or more times.
